(19) United States
(12) Patent Application Publication
(10) Pub. No.: US 2012/0027862 A1
(43) Pub. Date: Feb. 2, 2012

(54) Azelaic acid-comprising formulation with added pigment

(75) Inventors: Timm SCHMIDT, Berlin (DE); Thomas Zollner, Berlin (DE); Markus Friedrich, Oranienburg OT Lehnitz (DE)

(73) Assignee: INTENDIS GMBH, Berlin (DE)

(21) Appl. No.: 13/116,791

(22) Filed: May 26, 2011

(30) Foreign Application Priority Data
May 27, 2010 (DE) 102010021671.2

(51) Int. Cl. A61K 9/14; A61P 17/10; A61P 17/00; A61K 31/20; A61K 9/00

(52) U.S. Cl. 424/490; 424/63; 424/400

(57) ABSTRACT

The present patent application relates to an azelaic acid-comprising pharmaceutical composition, preferably an azelaic acid-comprising gel, with added pigment, which composition can be used, inter alia, for treating rosacea and acne, and also to a process for its preparation.

The present patent application relates to an azelaic acid-comprising pharmaceutical composition, preferably an azelaic acid-comprising gel, with added pigment, which composition can be used, inter alia, for treating rosacea and acne, and also to a process for its preparation.

### BACKGROUND AND PRIOR ART

It is known that topical formulations comprising azelaic acid can be used for treating certain dermatological disorders, for example rosacea and acne. Such compositions are described, inter alia, in the publications (DE 2817133 (preparation of azelaic acid), EP-A 0 305 407, EP-A 0 336 880, EP-A 1 032 379). These products can be present in various pharmaceutical forms, for example as cream, gel, lotion or foam. In some of the preparations of the prior art, use is also made of azelaic acid derivatives, for example dialkyl esters. In addition, some of the compositions also comprise other pharmaceutically active substances such as, for example, metronidazole (WO 2004/108143) or retinoids (WO 2007/082780).

For the treatment of hyperpigmented dermatoses, the publication DE 2817133 A1 describes, inter alia, a cream comprising azelaic acid and titanium dioxide. However, the subject of this publication is not the therapy of inflammatory and vascular processes such as, for example, acne or rosacea. The publication DE 19857491A1 describes a skin protection preparation for skin which tends to have impurities, which preparation comprises an added covering pigment or titanium dioxide as UV filter and azelaic acid as dermatologically active compound. The publication WO 95/25499 A1 describes a pulverulent cosmetic preparation which is active against acne and comprises titanium dioxide-coated mica and salicylic acid. However, topical preparations such as creams, gels, lotions or foams are not mentioned. Only solid pulverulent preparations based on silicones are disclosed. Furthermore, there is not disclosure of any specific azelaic acid-comprising formulation. The publication WO 2007/083174 A1 discloses pigments which are exclusively stoichiometric conjugates of inorganic pigment and an active compound for acne. The publication WO 2007/099398 A2 describes compositions consisting of metasilicates, carbonates, glyconates and one or more sulphates. These compositions may additionally comprise other active compounds, inter alia azelaic acid. Specific azelaic acid-comprising preparations are not disclosed.

Rosacea and acne are chronic skin disorders associated with skin reddening, in particular of the face. Rosacea is a common inflammatory-vascular skin disorder characterized by clinical symptoms such as erythema, flushing, teleangiectasias and/or inflammatory papules and pustules. In particular during the initiation of treatment, but also later on, the cosmetic problem of permanent facial erythema is an essential factor. Frequently, patients perceive this as an optical flaw. As a consequence, affected patients often tend to cover these reddenings cosmetically using, for example, covering sticks.

However, it is most unclear whether there are any interactions between the therapy with azelaic acid-comprising formulations and these cosmetics. Dermatologists can not exclude that such cosmetics may have an unfavourable effect on the course of the therapy.

There is therefore a need to provide novel azelaic acid-comprising compositions which allow a therapy of rosacea and acne and at the same time solve the individual cosmetic problems, without having an unfavourable effect on the course of the therapy.

Accordingly, it is an object of the present invention to provide stable azelaic acid-comprising formulations, in particular with proven active compound concentrations, for example from 10 to 25% by weight of azelaic acid, in particular 15% by weight or else 20% by weight, of azelaic acid, for the therapy of rosacea and acne, and at the same time to solve the individual cosmetic problems without any unfavourable effect on the course of the therapy. In particular, relatively large amounts of pigment, which may affect stability, activity or else the compatibility of the overall formulation, should be avoided.

### SUMMARY OF THE INVENTION

It has now been found that the addition of inorganic pigments to azelaic acid-comprising formulations resolves the problems described at the outset. Surprisingly, the combinations of azelaic acid and interference pigment showed improved antiinflammatory action compared to pure azelaic acid.

Furthermore, it has been found that stable pigment-comprising azelaic acid formulations can be prepared by a certain process. Here, the order of the process steps is important. In the process according to the invention, particular care has to be taken to avoid any rise in temperature during or after the addition.

Accordingly, the present invention comprises

Pharmaceutical composition, comprising 1-30% azelaic acid or an azelaic acid derivative and 0.01-10% of an interference pigment, characterized in, that the interference pigment consists of a phyllosilicate (KH₂(AlSiO₄)₃, derived from the mica group of silicates) which is coated with titanium dioxide, where phyllosilicate and titanium dioxide are present in a ratio of from 0.75:1 to 1:0.75. Especially the interference pigment comprises from 39 to 51 weight-% of potassium aluminium silicate and from 49 to 61 weight.-% titanium dioxide TiO₂.

Accordingly, the present invention comprises
1. Pharmaceutical composition, comprising 1-30% azelaic acid or an azelaic acid derivative and 0.01-10% of an interference pigment.
2. Pharmaceutical composition according to claim 1, characterized in that it is a gel.
3. Pharmaceutical composition according to claim 1, characterized in that the interference pigment is a titanium dioxide-coated silicate.
4. Pharmaceutical composition according to claim 1, characterized in that the interference pigment consists of a phyllosilicate (KH₂(AlSiO₄)₃, mica) which is coated with titanium dioxide,
   where phyllosilicate and titanium dioxide are present in a ratio of from 0.75:1 to 1:0.75, where 80% of the particles have a particle size in the range of 10-60 μm and where the mean particle diameter (D₅₀) is 18-25 μm.
5. Pharmaceutical composition according to claim 1 in the form of a gel, characterized by the following composition:

| | | |
|---|---|---|
| azelaic acid | 1.00-30.00% | by weight |
| mono/diglycerides | 0.00-3.00% | by weight |
| cetostearyl alcohol | 0.00-3.00% | by weight |
| PEG-40 stearate | 0.00-5.00% | by weight |
| medium-chain triglycerides | 0.00-15.00% | by weight |
| polysorbate 80 | 0.00-3.00% | by weight |
| lecithin | 0.00-10.00% | by weight |
| mono/diglycerides/cetostearyl alcohol/ | 0.00-10.00% | by weight |
| cetyl palmitate/cocoglycerides | | |
| mono/diglycerides/ | 0.00-10.00% | by weight |
| PEG-30 glyceryl stearate | | |
| cetearyl octanoate | 0.00-3.00% | by weight |
| methylcellulose | 0.00-1.00% | by weight |
| xanthan gum | 0.00-1.00% | by weight |
| Carbopol 980 | 0.50-2.00% | by weight |
| Carbopol 971 | 0.00-2.00% | by weight |
| water | 30.00-95.00% | by weight |
| dimethyl isosorbide | 0.00-15.00% | by weight |
| propylene glycol | 5.00-15.00% | by weight |
| glycerol 85% | 0.00-15.00% | by weight |
| benzoic acid | 0.05-1.00% | by weight |
| disodium EDTA | 0.00-0.50% | by weight |
| interference pigment | 0.01-10.00% | by weight |
| Candurin ® “Green Shimmer” | | |
| sodium hydroxide | 0.00-1.00% | by weight. |

6. Pharmaceutical composition according to claim 1 in the form of a cream, characterized by the following composition:

| | | |
|---|---|---|
| azelaic acid | 1.00-30.00% | by weight |
| mono/diglycerides | 0.00-3.00% | by weight |
| cetostearyl alcohol | 0.00-3.00% | by weight |
| PEG-40 stearate | 0.00-5.00% | by weight |
| medium-chain triglycerides | 0.00-15.00% | by weight |
| polysorbate 80 | 0.00-3.00% | by weight |
| lecithin | 0.00-10.00% | by weight |
| mono/diglycerides/cetostearyl alcohol/ | 0.00-10.00% | by weight |
| cetyl palmitate/cocoglycerides | | |
| mono/diglycerides/ | 0.00-10.00% | by weight |
| PEG-30 glyceryl stearate | | |
| cetearyl octanoate | 0.00-3.00% | by weight |
| methylcellulose | 0.00-1.00% | by weight |
| xanthan gum | 0.00-1.00% | by weight |
| Carbopol 980 | 0.50-2.00% | by weight |
| Carbopol 971 | 0.00-2.00% | by weight |
| water | 30.00-95.00% | by weight |
| dimethyl isosorbide | 0.00-15.00% | by weight |
| propylene glycol | 5.00-15.00% | by weight |
| glycerol 85% | 0.00-15.00% | by weight |
| benzoic acid | 0.05-1.00% | by weight |
| disodium EDTA | 0.00-0.50% | by weight |
| interference pigment | 0.01-10.00% | by weight |
| Candurin ® “Green Shimmer” | | |
| sodium hydroxide | 0.00-1.00% | by weight |

7. Pharmaceutical composition according to claim 1 in the form of a lotion, characterized by the following composition:

| | | |
|---|---|---|
| azelaic acid | 1.00-30.00% | by weight |
| mono/diglycerides | −0.00-3.00% | by weight |
| cetostearyl alcohol | 0.00-3.00% | by weight |
| PEG-40 stearate | 0.00-5.00% | by weight |
| medium-chain triglycerides | 0.00-15.00% | by weight |
| polysorbate 80 | 0.00-3.00% | by weight |
| lecithin | 0.00-10.00% | by weight |
| mono/diglycerides/cetostearyl alcohol/ | 0.00-10.00% | by weight |
| cetyl palmitate/cocoglycerides | | |
| mono/diglycerides/ | 0.00-10.00% | by weight |
| PEG-30 glyceryl stearate | | |
| cetearyl octanoate | 0.00-3.00% | by weight |
| methylcellulose | 0.00-1.00% | by weight |
| xanthan gum | 0.00-1.00% | by weight |
| Carbopol 980 | 0.00-2.00% | by weight |
| Carbopol 971 | 0.50-2.00% | by weight |
| water | 30.00-95.00% | by weight |
| dimethyl isosorbide | 0.00-15.00% | by weight |
| propylene glycol | 5.00-15.00% | by weight |
| glycerol 85% | 0.00-15.00% | by weight |
| benzoic acid | 0.05-1.00% | by weight |
| disodium EDTA | 0.00-0.50% | by weight |
| interference pigment | 0.01-10.00% | by weight |
| Candurin ® “Green Shimmer” | | |
| (mica + TiO₂) | | |
| sodium hydroxide | 0.00-1.00% | by weight. |

8. Pharmaceutical composition according to claim 1 in the form of a foam, characterized by the following composition:

| | | |
|---|---|---|
| azelaic acid | 1.00-30.00% | by weight |
| mono/diglycerides | 0.00-3.00% | by weight |
| cetostearyl alcohol | 0.00-3.00% | by weight |
| PEG-40 stearate | 0.00-5.00% | by weight |
| medium-chain triglycerides | 0.00-15.00% | by weight |
| polysorbate 80 | 0.00-3.00% | by weight |
| lecithin | 0.00-10.00% | by weight |
| mono/diglycerides/cetostearyl alcohol/ | 0.00-10.00% | by weight |
| cetyl palmitate/cocoglycerides | | |
| mono/diglycerides/ | 0.00-10.00% | by weight |
| PEG-30 glyceryl stearate | | |
| cetearyl octanoate | 0.00-3.00% | by weight |
| methylcellulose | 0.00-1.00% | by weight |
| xanthan gum | 0.00-1.00% | by weight |
| Carbopol 980 | 0.00-2.00% | by weight |
| Carbopol 971 | 0.00-2.00% | by weight |
| water | 30.00-95.00% | by weight |
| dimethyl isosorbide | 0.00-15.00% | by weight |
| propylene glycol | 5.00-15.00% | by weight |
| glycerol 85% | 0.00-15.00% | by weight |
| benzoic acid | 0.05-1.00% | by weight |
| disodium EDTA | 0.00-0.50% | by weight |
| interference pigment | 0.01-10.00% | by weight |
| Candurin ® “Green Shimmer” | | |
| sodium hydroxide | 0.00-1.00% | by weight |

9. Use of a composition according to any of claims 1-8 for preparing a medicament for the therapy of rosacea and acne.
10. Use of a composition according to any of claims 1-8 for the cosmetic covering of skin erythema caused by rosacea and/or acne.
11. Use of a composition according to any of claims 1-8 as sunscreen suitable for skin erythema caused by rosacea and/or acne.
12. Medicament for the therapy of rosacea and acne, comprising a composition according to any of claims 1-8.
13. Process for preparing a composition according to any of claims 1-8, characterized by the following steps:
   a) mixing of the hydrophilic components,
   b) heating to 60°-80° C.,
   c) dissolving of preservatives and complex formers in the warm mixture,
   d) addition and dispersion of the pigment or the pigments by intensive homogenization,
   e1) addition and dispersion of the gel former or the gel formers by another homogenization,
   e2) heating of the oil phase to 60°-80° C., in parallel to step e1
   f) addition of the oil phase from e2 to the prepared dispersion from e1 at 60°-80° C., and another homogenization of the overall formulation,
   g) cooling to 20°-35° C.,
   h) addition of azelaic acid to the dispersion (cooled to 20°-35° C.), with avoidance of heating and/or sheering,
   i) pH-stabilization with formation of the gel by addition of sodium hydroxide solution. (nominal pH: 4.6-5.0)

### BRIEF DESCRIPTION OF THE DRAWINGS

FIG. 1 shows the absorption behaviour of the formulations in the examples.

### DETAILED DESCRIPTION OF THE INVENTION

In one embodiment, the formulations according to the invention comprise 1-30% azelaic acid or an azelaic acid derivative, 0.01-10% of an interference pigment and at least one gelling agent selected from the following group: methylcellulose, xanthan gum, crosslinked polyacrylic acid polymers such as, for example, Carbopol 980 (CAS No. 9063-87-0) or Carbopol 971.

In another embodiment, the formulations according to the invention comprise 1-30% azelaic acid or an azelaic acid derivative, 0.01-10% of an interference pigment and at least one gelling agent selected from the following group: methylcellulose, xanthan gum, crosslinked polyacrylic acid polymers such as, for example, Carbopol 980 (CAS No. 9063-87-0) or Carbopol 971 and at least one lipid/stabilizer selected from the following group: mono- and diglycerides, cetostearyl alcohol, PEG-40 stearate, medium-chain triglycerides, polysorbate 80, lecithin, mono- and diglycerides/cetostearyl alcohol/cetyl palmitate/cocoglycerides, mono- and diglycerides/PEG-30 glyceryl stearate, cetearyl octanoate.

In a further embodiment, the formulations according to the invention comprise 1-30% azelaic acid or an azelaic acid derivative, 0.01-10% of an interference pigment and at least one hydrophilic component selected from the following group: water, dimethyl isosorbide, propylene glycol, and glycerol (85%).

In another embodiment, the formulations according to the invention comprise 1-30% azelaic acid or an azelaic acid derivative, 0.01-10% of an interference pigment and 0-5%, especially 0-2% of a gelling agent, selected from the following group: methylcellulose, xanthan gum, crosslinked polyacrylic acid polymers such as, for example, Carbopol 980 (CAS No. 9063-87-0) or Carbopol 971. Gels comprise as gelling agent 0-2% crosslinked polyacrylic acid, preferably 0.5-1%. Foam compositions comprise as gelling agent 0-2% methylcellulose and or Xanthan gum,

In another embodiment, the formulations according to the invention comprise 1-30% azelaic acid or an azelaic acid derivative, 0.01-10% of an interference pigment and 0-5%, especially 0-2% of a gelling agent, selected from the following group: methylcellulose, xanthan gum, crosslinked polyacrylic acid polymers such as, for example, Carbopol 980 (CAS No. 9063-87-0) or Carbopol 971 furthermore 0-3% polysorbates and furthermore 0-10%, preferably 0-5%, preferably 1% soy bean lecithin or lecithin. Gels comprise as gelling agent 0-2% crosslinked polyacrylic acid, preferably 0.5-1%. Foam compositions comprise as gelling agent 0-2% methylcellulose and or Xanthan gum,

In another embodiment, the formulations according to the invention comprise 1-30% azelaic acid or an azelaic acid derivative, 0.01-10% of an interference pigment and 0-5%, especially 0-2% of a gelling agent, selected from the following group: methylcellulose, xanthan gum, crosslinked polyacrylic acid polymers such as, for example, Carbopol 980 (CAS No. 9063-87-0) or Carbopol 971 furthermore 0-3% polysorbates and furthermore 0-10%, preferably 0-5%, preferably 1% soy bean lecithin and lecithin and optionally furthermore 0-15%, preferably 0.5-5%, preferably 0-2%, preferably 1% medium chain triglycerides. Gels comprise as gelling agent 0-2% crosslinked polyacrylic acid, preferably 0.5-1%. Foam compositions comprise as gelling agent 0-2% methylcellulose and or Xanthan gum,

In another embodiment, the formulations according to the invention comprise 1-30% azelaic acid or an azelaic acid derivative, 0.01-10% of an interference pigment and 0-5%, especially 0-2% of a gelling agent, selected from the following group: methylcellulose, xanthan gum, crosslinked polyacrylic acid polymers such as, for example, Carbopol 980 (CAS No. 9063-87-0) or Carbopol 971 furthermore 0-3% polysorbates and furthermore 0-10%, preferably 0-5%, preferably 1% soy bean lecithin and lecithin and optionally furthermore 0-15%, preferably 0.5-5%, preferably 0-2%, preferably 1% medium chain triglycerides and optionally furthermore 5-15%, preferably 5-13, preferably 10-13% preferably 11-13% propylene glycol. Gels comprise as gelling agent 0-2% crosslinked polyacrylic acid, preferably 0.5-1%. Foam compositions comprise as gelling agent 0-2% methylcellulose and or Xanthan gum,

Preferred pigments according to the invention are phyllosilicates, in particular interference pigments. Interference pigments have the property to reflect incident light in a different colour. These are silicates coated with oxides of titanium, iron or chromium. The silicate used according to the invention is in particular potassium aluminium silicate of the structure KH₂(AlSiO₄)₃ coated with titanium dioxide, namely in a ratio of from 0.75:1 to 1:0.75. Titanium dioxide-coated potassium aluminium silicate (titanium dioxide-coated potassium aluminium silicate) is a mica-based pearl-effect colorant used for colouring food and medicaments. It is an interference pigment with a greenish shimmer which, by breaking/reflecting the light, is able to ameliorate erythemas.

It has to be assumed that the addition of the pigments mentioned above to the azelaic acid-comprising formulations has no negative effect on the course of the therapy but rather, in multifarious ways, has a positive effect. The use of the pigments furthermore achieves an optically very noticeable reduction of the skin reddening, such that the patients are stabilized psychically: the effect of the “disappearance” of the erythemas gives the patients the feeling that no further cosmetics are required for covering. In addition, it has been found that the formulations according to the invention can be used for very different skin types and skin tones, whereas conventional cosmetics (such as covering sticks and the like) have to be adapted individually to the respective skin tone or skin type. As a result, the patients rate the preparation markedly higher, which leads to better patient compliance and in turn to better results of the therapy. In this respect, it has been found in particular that the formulations according to the invention are preferred especially by men who for their part are likewise affected by rosacea/acne, since men, too, are aware of the cosmetic problems of rosacea/acne but at the same time are reluctant to use covering cosmetics. By using the formulation according to the invention, the cosmetic problem of the male patients is solved without additional cosmetics being required.

In addition, it has been found that the formulation according to the invention also acts as a light screen. In this sense, it also acts as UV protection for the affected skin. This has the benefit that the skin regions in question are protected against additional irritation by UV rays. Furthermore, there is no need to employ corresponding sun screen formulations unless there is intensive irradiation (such as, for example, on the beach, close to the equator or at high altitudes).

The formulations according to the invention can be present as a gel, as a cream, as a lotion or as a foam, with gels being preferred according to the invention. In any case, the therapeutically active ingredient used is azelaic acid or an azelaic acid derivative (for example a mono- or dialkyl ester having 1 to 4 carbon atoms). The azelaic acid is used in an amount of from 1 to 30% by weight, preferably from 10 to 25% by weight, where 15% by weight are preferred for a gel and 20% by weight are preferred for a cream.

The lipids/stabilizers used are, for example, the following products: mono- and diglycerides, cetostearyl alcohol, PEG-40 stearate, medium-chain triglycerides, polysorbate 80, lecithin, mono- and diglycerides/cetostearyl alcohol/cetyl palmitate/cocoglycerides, mono- and diglycerides/PEG-30 glyceryl stearate, cetearyl octanoate.

The gelling agent used is, for example, methylcellulose, xanthan gum, crosslinked polyacrylic acid polymers such as, for example, Carbopol 980 (CAS No. 9063-87-0) or Carbopol 971.

Hydrophilic components are water, dimethyl isosorbide, propylene glycol, and glycerol (85%).

The preservative employed is, for example, benzoic acid.

The complex former used is, for example, disodium EDTA.

The pigments are in particular green interference pigments such as, for example, Candurin® “Green Shimmer”, which is commercially available from Merck KGaA, Darmstadt, Germany and consists of potassium aluminium silicate KH₂(AISiO₄)₃ (CAS 12001262) coated with titanium dioxide TiO₂(CAS 13463677). Here, the potassium aluminium silicate and the titanium dioxide are present in a ratio of from 0.75:1 to 1:0.75, where 80% of the particles should have a particle size in the range of 10-60 μm (determined by laser diffraction using, for example, a Mastersizer Malvern 2000). The mean particle diameter (D₅₀) should be 18-25 μm. Especially the interference pigment comprises from 39 to 51 weight-% of potassium aluminium silicate and from 49 to 61 weight.-% titanium dioxide TiO₂. Further details can be found in the manufacturers specification. The formulations according to the invention comprise 0.01-10.00% by weight of pigment. Preferably, they comprise 0.1-8% by weight, with preference 0.5-6% by weight, in particular 1-5% by weight, more preferably 3% by weight.

It is also possible to use silicate coated with Iron Oxide Red (CAS 1309371), Iron Oxide Yellow (CAS 51274001, Iron Oxide Black (CAS 1317619) or Chromium Oxide Green (CAS 1308389), or to use pure oxides of iron and chromium and mixtures of these, depending on the desired colour result and covering performance.

However, if the skin reddening is to be reduced in a manner which is optically as neutral as possible, it is recommended in particular to use an above-described titanium dioxide-coated silicate, because it reflects the light in the complementary colour green but does not colour the formulation itself, nor face and clothing.

To stabilize the pH, use is made in particular of sodium hydroxide solution.

The activity optimum of azelaic acid is, naturally, in the acidic range, and colorants are known to be unstable or prone to discolouration outside the neutral pH range of 7; however, surprisingly, it was nevertheless possible to prepare colour-stable formulations with the selected pigments and an acidic pH (nominal pH: 4.6-5.0).

Tables 1 to 4 show exemplary compositions of formulations according to the invention, however, these are not to be understood to be limiting. The quantities are stated in % by weight. The preferred compositions are in each case stated in brackets.

TABLE 1
Gel formulation

| Function | Ingredient | AzA Gel | |
|---|---|---|---|
| Active | azelaic acid | 1.00-30.00 | (15.00) |
| compound | | | |
| Lipids/ | mono/diglycerides | 0.00-3.00 | (0.00) |
| stabilizers | cetostearyl alcohol | 0.00-3.00 | (0.00) |
| | PEG-40 stearate | 0.00-5.00 | (0.00) |
| | medium-chain triglycerides | 0.00-15.00 | (1.00) |
| | polysorbate 80 | 0.00-3.00 | (1.50) |
| | lecithin | 0.00-10.00 | (1.00) |
| | mono/diglycerides/cetostearyl | 0.00-10.00 | (0.00) |
| | alcohol/cetyl palmitate/ | | |
| | cocoglycerides | | |
| | mono/diglycerides/ | 0.00-10.00 | (0.00) |
| | PEG-30 glyceryl stearate | | |
| | cetearyl octanoate | 0.00-3.00 | (0.00) |
| Gelling agent | methylcellulose | 0.00-1.00 | (0.00) |
| | xanthan gum | 0.00-1.00 | (0.00) |
| | Carbopol 980 | 0.50-2.00 | (1.00) |
| | Carbopol 971 | 0.50-2.00 | (0.00) |
| Hydrophilic | water | 30.00-95.00 | (68.10) |
| components | dimethyl isosorbide | 0.00-15.00 | (0.00) |
| | propylene glycol | 5.00-15.00 | (12.00) |
| | glycerol 85% | 0.00-15.00 | (0.00) |
| Preservative | benzoic acid | 0.05-1.00 | (0.10) |
| Complex | disodium EDTA | 0.00-0.50 | (0.10) |
| former | | | |
| Pigment | interference pigment | 0.01-10.00 | (3.00) |
| | Candurin ® “Green Shimmer” | | |
| pH-Stabilizers | sodium hydroxide | 0.00-1.00 | (0.20) |

TABLE 2
Cream formulation

| Function | Ingredient | AzA Cream | |
|---|---|---|---|
| Active compound | azelaic acid | 1.00-30.00 | (20.00) |
| Lipids/Stabilizers | mono/diglycerides | 0.00-3.00 | (0.00) |
| | cetostearyl alcohol | 0.00-3.00 | (0.00) |
| | PEG-40 stearate | 0.00-5.00 | (0.00) |
| | medium-chain triglycerides | 0.00-15.00 | (0.00) |
| | polysorbate 80 | 0.00-3.00 | (0.00) |
| | lecithin | 0.00-10.00 | (0.00) |
| | mono/diglycerides/cetostearyl | 0.00-10.00 | (7.00) |
| | alcohol/cetyl palmitate/ | | |
| | cocoglycerides | | |
| | mono/diglycerides/ | 0.00-10.00 | (5.00) |
| | PEG-30 glyceryl stearate | | |
| | cetearyl octanoate | 0.00-3.00 | (1.50) |
| Gelling agent | methylcellulose | 0.00-1.00 | (0.00) |
| | xanthan gum | 0.00-1.00 | (0.00) |
| | Carbopol 980 | 0.50-2.00 | (0.00) |
| | Carbopol 971 | 0.50-2.00 | (0.00) |
| Hydrophilic | water | 30.00-95.00 | (50.80) |
| components | | | |
| | dimethyl isosorbide | 0.00-15.00 | (0.00) |
| | propylene glycol | 5.00-15.00 | (12.50) |
| | glycerol 85% | 0.00-15.00 | (1.50) |
| Preservative | benzoic acid | 0.05-1.00 | (0.20) |
| Complex former | disodium EDTA | 0.00-0.50 | (0.00) |
| Pigment | interference pigment | 0.01-10.00 | (3.00) |
| | Candurin ® “Green | | |
| | Shimmer” | | |
| pH-Stabilizers | sodium hydroxide | 0.00-1.00 | (0.20) |

TABLE 3
Lotion formulation

| Function | Ingredient | AzA Lotion | |
|---|---|---|---|
| Active compound | azelaic acid | 1.00-30.00 | (15.00) |
| Lipids/Stabilizers | mono/diglycerides | −0.00-3.00 | (0.00) |
| | cetostearyl alcohol | 0.00-3.00 | (0.00) |
| | PEG-40 stearate | 0.00-5.00 | (0.00) |
| | medium-chain triglycerides | 0.00-15.00 | (1.00) |
| | polysorbate 80 | 0.00-3.00 | (1.50) |
| | lecithin | 0.00-10.00 | (1.00) |
| | mono/diglycerides/cetostearyl | 0.00-10.00 | (0.00) |
| | alcohol/cetyl palmitate/ | | |
| | cocoglycerides | | |
| | mono/diglycerides/ | 0.00-10.00 | (0.00) |
| | PEG-30 glyceryl stearate | | |
| | cetearyl octanoate | 0.00-3.00 | (0.00) |
| Gelling agent | methylcellulose | 0.00-1.00 | (0.00) |
| | xanthan gum | 0.00-1.00 | (0.00 |
| | Carbopol 980 | 0.50-2.00 | (0.00) |
| | Carbopol 971 | 0.50-2.00 | (1.00) |
| Hydrophilic | water | 30.00-95.00 | (68.10) |
| components | | | |
| | dimethyl isosorbide | 0.00-15.00 | (0.00) |
| | propylene glycol | 5.00-15.00 | (12.00) |
| | glycerol 85% | 0.00-15.00 | (0.00) |
| Preservative | benzoic acid | 0.05-1.00 | (0.10) |
| Complex former | disodium EDTA | 0.00-0.50 | (0.10) |
| Pigment | interference pigment | 0.01-10.00 | (3.00) |
| | Candurin ® “Green | | |
| | Shimmer” | | |
| pH-Stabilizers | sodium hydroxide | 0.00-1.00 | (0.20) |

TABLE 4
Foam formulation

| Function | Ingredient | AzAFoam | |
|---|---|---|---|
| Active | azelaic acid | 1.00-30.00 | (15.00) |
| compound | | | |
| Lipids/ | mono/diglycerides | 0.00-3.00 | (0.54) |
| Stabilizers | | | |
| | cetostearyl alcohol | 0.00-3.00 | (1.09) |
| | PEG-40 stearate | 0.00-5.00 | (2.83) |
| | medium-chain triglycerides | 0.00-15.00 | (10.87) |
| | polysorbate 80 | 0.00-3.00 | (0.98) |
| | lecithin | 0.00-10.00 | (1.00) |
| | mono/diglycerides/cetostearyl | 0.00-10.00 | (0.00) |
| | alcohol/cetyl palmitate/ | | |
| | cocoglycerides | | |
| | mono/diglycerides/ | 0.00-10.00 | (0.00) |
| | PEG-30 glyceryl stearate | | |
| | cetearyl octanoate | 0.00-3.00 | (0.00) |
| Gelling agent | methylcellulose | 0.00-1.00 | (0.11) |
| | xanthan gum | 0.00-1.00 | (0.27) |
| | Carbopol 980 | 0.50-2.00 | (0.00) |
| | Carbopol 971 | 0.50-2.00 | (0.00) |
| Hydrophilic | water | 30.00-95.00 | (51.45) |
| components | | | |
| | dimethyl isosorbide | 0.00-15.00 | (5.44) |
| | propylene glycol | 5.00-15.00 | (10.87) |
| | glycerol 85% | 0.00-15.00 | (0.00) |
| Preservative | benzoic acid | 0.05-1.00 | (0.10) |
| Complex former | disodium EDTA | 0.00-0.50 | (0.00) |
| Pigment | interference pigment | 0.01-10.00 | (3.00) |
| | Candurin ® “Green | | |
| | Shimmer” (mica + TiO₂) | | |
| pH-Stabilizers | sodium hydroxide | 0.00-1.00 | (0.20) |

To prepare a foam, the composition shown in Table 4 is mixed with an additional 1-100% by weight (preferably 2-10% by weight) of a propellant and filled into a container (for example a spray can) having an exit nozzle. Preferred propellants are substances which are present as gases at room temperature, but which can be liquefied under pressure. Examples of such propellants are hydrocarbons (such as propane, butane, isobutane), fluorinated alkanes (such as heptafluoropropane or tetrafluoroethane), dimethyl ether or mixtures thereof. Processes for preparing such foams are described in the literature and do not require any further illustration here. To be on the safe side, it should only be mentioned here that the composition described in Table 4 does, of course, add up to 100% by weight. The propellant is added in addition thereto.

### Preparation

The formulations according to the invention are prepared by the process described below:

Initially, the hydrophilic components of the formulations are mixed and heated to 60°-80° C., preferably 70° C. The preservatives and complex formers are dissolved in this mixture.

The pigment or the pigments are then dispersed by intensive homogenization. This is followed by dispersing the gel former or the gel formers in another homogenization. In parallel, the oil phase is likewise heated to 60°-80° C., preferably 70° C., and added to the dispersion prepared as described above, and the overall formulation is homogenized once more.

With stirring, the mixture is then cooled to 20°-35° C., preferably 30° C.

The azelaic acid is added to the cooled dispersion, with neither warming nor sheering.

Finally, the pH is stabilized by addition of sodium hydroxide solution (nominal pH: 4.6-5.0), with formation of the gel portion.

The stated temperatures are to be understood as preferred ranges. The components may also be mixed at temperatures of 55-85° C.

The oil phase may also be at temperatures of 55-85° C.

It has to be emphasized here that it is not possible to prepare the formulations according to the invention, in particular gels according to the invention, by standard methods. The simple addition of pigments to formulations comprising azelaic acid leads to instabilities of these formulations. In the process according to the invention, particular care has to be taken that there is no increase in temperature during or after the addition of the azelaic acid. The instabilities observed are probably due to the fact that at elevated temperature some or all of the azelaic acid goes into solution and then crystallizes again, the pigments serving as seed crystals. Unfortunately, the formulations formed in this manner are unstable. The preparation process described above solves these problems since the azelaic acid is added to the cooled formulation, and there is therefore no partial or complete dissolution of the azelaic acid. Furthermore, the addition of the azelaic acid is very late in the preparation process, so that there is no energetic stress on the azelaic acid owing to stirring. Finally, the viscosity of the formulation is only produced by the neutralization of the carbomer at the end of the preparation process, which facilitates the homogeneous incorporation both of the pigments and the azelaic acid. For the person skilled in the art, the problems, described above, in the preparation of the formulations according to the invention and their resolution by the process according to the invention were not obvious.

### EXAMPLES

The invention is illustrated by the examples below, but these examples are not to be construed to be limiting. Example 1 is the reference formulation without added pigment.

| | Example 1 | Example 2 | Example 3 |
|---|---|---|---|
| azelaic acid | 15% | 15% | 15% |
| benzoic acid | 0.1% | 0.1% | 0.1% |
| disodium EDTA | 0.1% | 0.1% | 0.1% |
| Carbopol 980 | 1% | 1% | 1% |
| propylene glycol | 12% | 12% | 12% |
| polysorbate 80 | 1.5% | 1.5% | 1.5% |
| medium-chain triglycerides | 1% | 1% | 1% |
| lecithin | 1% | 1% | 1% |
| Candurin ® “Green Shimmer” | 0% | 1.5% | 3% |
| sodium hydroxide | 0.2%. | 0.2%. | 0.2% |

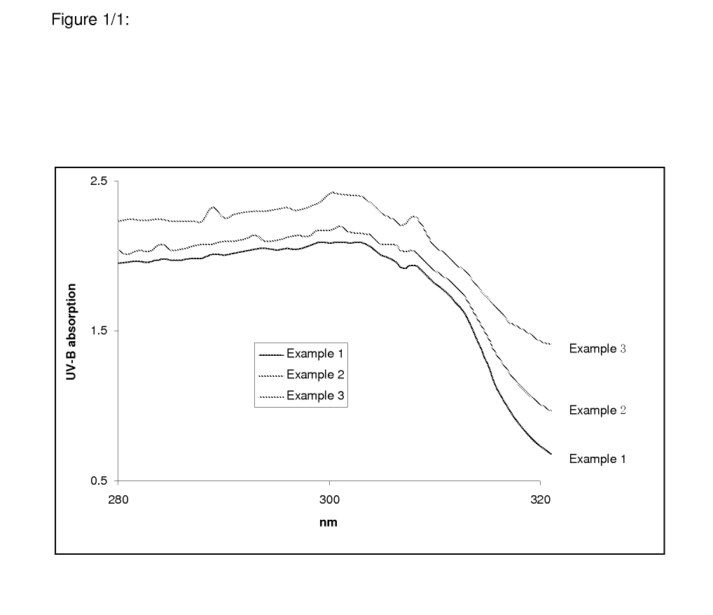

A formulation according to the invention in the form of a gel is prepared by the process described above. The gel comprises 15% by weight azelaic acid and up to 3% Candurin® “Green Shimmer” pigment. When measuring the absorption of the UV-B radiation spectrum responsible for sun damage of the skin (280-320 nm), it was surprisingly found that all three formulations have an absorption behaviour which would be desirable for a sunscreen product (see FIG. 1). In each case, the absorption peak is in the activity maximum of the UV-B rays responsible for erythema formation (“sunburn”) (300 nm). Depending on the concentration of the pigment, the absorption of harmful UV-B rays could be increased even more. Accordingly, the combination of azelaic acid with the pigment surprisingly shows a synergistic effect with respect to the protection against UV-B radiation.

Moreover, owing to the pharmaceutical properties, which, with the exception of the colouration, are identical to the preparation without pigment, the formulation according to the invention shows therapeutic results which are comparable to or better than Finacea®. At the same time, the volunteers indicated that they prefer the gel according to the invention, owing to the improved cosmetic properties and the resulting marked optical reduction of skin reddening. This preference leads to a better compliance with the therapy protocol, which in turn results in a more rapid healing of the erythemas.

In a further test, the impact of the pigment used on the known effects of azelaic acid is investigated with respect to the expression of the cytokines IL-1β, IL-6 and TNF-alpha. In addition to these cytokines, it is also intended to examine the influence on the chemotactic factor MCP-1 (monocytic chemotactic protein-1), which is of general importance for cutaneous inflammatory reactions, and on the factor VEGF/VPF (vascular endothelial growth factor/vascular permeability factor), which is important for rosacea pathogenesis.

### Materials and Methods

HaCaT cells originate from a spontaneously immortalized permanent human keratinocyte cell line established by long-term cultivation of adult human keratinocytes under reduced calcium concentration and elevated temperature (Boukamp et al. J Cell Biol 106:761-771, 1988). HaCaT cells were propagated in culture medium. This consists of 88 ml DMEM/Ham's F12 w/o glutamine, 10 ml foetal calf serum, 1 ml penicillin/streptomycin solution with 10,000 units/ml and 1 ml of glutamine. The cells were expanded in culture bottles, detached with trypsin/EDTA (0.25% trypsin, 1 mMol EDTA×4 Na) in Hank's Buffered Saline without Ca/Mg at about 80% confluence and transferred into Petri dishes for the experiments.

HaCaT cells were sown in 6-well or 12-well plates at a cell number of 3×10⁵ and 1.5×10⁵ cells/well, respectively. The cells were cultivated to about 80% confluence in culture medium. 2 h prior to the intended UV irradiation, the cells were incubated with the above-mentioned substances. Immediately before the irradiation, the cells were washed 2× with PBS and irradiated therein. Irradiation was with UVB (spectrum: 280-360 nM, max. 320 nM) in a Waldmann-UVB source UV7002 at 100 mJ. After the UV irradiation, the cells were irradiated in culture medium in the presence and absence of the abovementioned substances. The cultures were stopped 24 h or 48 h after the UVB irradiation, and the supernatant was removed and centrifuged to remove debris and titanium dioxide-coated potassium aluminium silicate. All cultures were set up in triplicate.

The supernatants were frozen at −80° C. until further use. The protein concentration was determined using a commercial kit from Meso Scale Discovery, Gaithersburg, Md., USA. The test was carried out in accordance with the instructions of the manufacturer (www.mesoscale.com). After addition of the blocking solution to the plates precoated with scavenger antibodies, the samples were incubated for 1 h and then washed. Subsequently, diluent and 25 μl of the sample or the calibration solution were added, and the mixture was incubated for another 2 hours and washed again. After this time, the detection antibody was added for 1 h. The samples were then washed once more, 150 μl of read buffer were pipetted into the mixture and the electrochemiluminescence was measured in an MSD Sector plate reader. The experiment was carried out analogously to Mastrofrancesco et al. (Mastrofrancesco A, Ottaviani M, Aspite N, Cardinali G, Izzo E, Graupe K, Zouboulis C C, Camera E, Picardo M. Azelaic acid modulates the inflammatory response in normal human keratinocytes through PPARgamma activation. Exp Dermatol. 2010; 19:813-20) where the positive effect of azelaic acid on the UVB-induced inflammatory response of keratinocytes was demonstrated (Mastrofrancesco et al.). This paper shows that UVB irradiation results in an induction of the cytokines IL-1β, IL-6 and TNF-α. The following cell cultures were prepared:
unirradiated HaCaTs
irradiated HaCaTs (100 mJ)
irradiated HaCaTs (100 mJ) in the presence of azelaic acid,
irradiated HaCaTs (100 mJ) in the presence of azelaic acid and titanium dioxide-coated potassium aluminium silicate

The following reagents were used to treat the cells:
azelaic acid, Fluka, batch S3317131508B29, 20 mM
titanium dioxide-coated potassium aluminium silicate, Merck Darmstadt, batch W08012807X, 3%

The following cytokines/chemokines were analyzed:
IL-1β
IL-6
TNF-α
VEGF
MCP-1=CCL2

HaCaT cells were cultivated in triplicates, irradiated with 100 mJ UVB and then cultivated in the presence or absence of azelaic acid and, if appropriate, titanium dioxide-coated potassium aluminium silicate or the combination thereof for 24 or 48 h. The supernatants were then used to measure proinflammatory cytokines. The cytokines IL-1β (A), IL-6 (B), TNFα (C), VEGF (D) and MCP-1/CCL2 (E) were measured in the supernatant in triplicate after 24 (series I) and 48 h (series II) using Mesoscale. What is shown are the mean values and also the respective individual values of the protein determinations.

In the tables, the abbreviation Aza denotes azelaic acid and Green denotes the titanium dioxide-coated potassium aluminium silicate from Merck Darmstadt obtainable as Candurin® “Green Shimmer”, consisting of potassium aluminium silicate KH₂(AlSiO₄)₃ (CAS 12001262) coated with titanium dioxide TiO₂(CAS 13463677). Here, potassium aluminium silicate and titanium dioxide are present in a ratio of from 0.75:1 to 1:0.75, where for 80% of the particles the particle size should be in the range of 10-60 μm.

TABLE 1a
IL-1β in HACaT cell supernatants after 24 h in pg/l

| | | | Azelaic | Aza + |
|---|---|---|---|---|
| | Control | Control | acid | Green |
| | unirradiated | irradiated | irradiated | irradiated |
| | 24 h (n = 3) | 24 h (n = 3) | 24 h (n = 3) | 24 h (n = 3) |
| Mean | 8.3 | 4.6 | 5.8 | 1.0 |
| #1 | 5.4 | 3.9 | 3.1 | 0.7 |
| #2 | 12.3 | 4.6 | 3.1 | 0.5 |
| #3 | 7.2 | 5.2 | 11.3 | 2.0 |

TABLE 1b
IL-1β in HACaT cell supernatants after 48 h in pg/l

| | | | Azelaic | Aza + |
|---|---|---|---|---|
| | Control | Control | acid | Green |
| | unirradiated | irradiated | irradiated | irradiated |
| | 48 h (n = 3) | 48 h (n = 3) | 48 h (n = 3) | 48 h (n = 3) |
| Mean | 9.8 | 16.8 | 10.4 | 3.6 |
| #1 | 9.3 | 20.4 | 9.7 | 3.2 |
| #2 | 10.2 | 18.6 | 7.2 | 3.1 |
| #3 | 10.0 | 11.5 | 14.3 | 4.4 |

TABLE 1c
IL-6 in HACaT cell supernatants after 24 h in pg/l

| | | | | Aza + |
|---|---|---|---|---|
| | Control | Control | Azelaic acid | Green |
| | unirradiated | irradiated | irradiated | irradiated |
| | 24 h (n = 3) | 24 h (n = 3) | 24 h (n = 3) | 24 h (n = 3) |
| Mean | 39.3 | 33.8 | 44.3 | 27.9 |
| #1 | 35.0 | 34.9 | 37.4 | 31.7 |
| #2 | 36.8 | 32.8 | 24.7 | 23.6 |
| #3 | 46.0 | 33.7 | 70.9 | 28.4 |

TABLE 1d
IL-6 in HACaT cell supernatants after 48 h in pg/l

| | | | Azelaic | Aza + |
|---|---|---|---|---|
| | Control | Control | acid | Green |
| | unirradiated | irradiated | irradiated | irradiated |
| | 48 h (n = 3) | 48 h (n = 3) | 48 h (n = 3) | 48 h (n = 3) |
| Mean | 80.2 | 274.7 | 120.7 | 62.4 |
| #1 | 66.1 | 305.5 | 70.5 | 61.2 |
| #2 | 88.9 | 281.0 | 59.2 | 58.6 |
| #3 | 85.6 | 237.4 | 232.3 | 67.5 |

TABLE 1e
TNF-α in HACaT cell supernatants after 24 h in pg/l

| | | | | Aza + |
|---|---|---|---|---|
| | Control | Control | Azelaic acid | Titan. |
| | unirradiated | irradiated | irradiated | irradiated |
| | 24 h (n = 3) | 24 h (n = 3) | 24 h (n = 3) | 24 h (n = 3) |
| Mean | 13.1 | 8.0 | 7.2 | 1.6 |
| #1 | 10.9 | 6.8 | 6.7 | 1.0 |
| #2 | 14.5 | 6.6 | 7.6 | 0.3 |
| #3 | 14.0 | 10.7 | 21.63 | 3.4 |

TABLE 1f
TNF-α in HACaT cell supernatants after 48 h in pg/l

| | | | | Aza + |
|---|---|---|---|---|
| | Control | Control | Azelaic acid | Titan. |
| | unirradiated | irradiated | irradiated | irradiated |
| | 48 h (n = 3) | 48 h (n = 3) | 48 h (n = 3) | 48 h (n = 3) |
| Mean | 18.8 | 77.1 | 30.0 | 4.1 |
| #1 | 18.3 | 96.6 | 17.4 | 4.7 |
| #2 | 19.8 | 90.6 | 14.4 | 4.3 |
| #3 | 18.1 | 44.1 | 58.0 | 3.3 |

TABLE 1g
VGEF in HACaT cell supernatants after 24 h in pg/l

| | | | | Aza + |
|---|---|---|---|---|
| | Control | Control | Azelaic acid | Titan. |
| | unirradiated | irradiated | irradiated | irradiated |
| | 24 h (n = 3) | 24 h (n = 3) | 24 h (n = 3) | 24 h (n = 3) |
| Mean | 1110.5 | 1056.1 | 692.4 | 255.8 |
| #1 | 1161.2 | 1107.1 | 751.4 | 300.2 |
| #2 | 1050.4 | 1071.3 | 633.4 | 216.8 |
| #3 | 1120.0 | 989.8 | 1694.59 | 250.3 |

TABLE 1h
VGEF in HACaT cell supernatants after 48 h in pg/l

| | | | | Aza + |
|---|---|---|---|---|
| | Control | Control | Azelaic acid | Titan. |
| | unirradiated | irradiated | irradiated | irradiated |
| | 48 h (n = 3) | 48 h (n = 3) | 48 h (n = 3) | 48 h (n = 3) |
| Mean | 3059.5 | 6363.0 | 3999.0 | 1208.6 |
| #1 | 3158.6 | 7447.1 | 3239.6 | 1284.2 |
| #2 | 3038.0 | 7593.4 | 2945.2 | 1195.9 |
| #3 | 2982.0 | 4048.5 | 5812.0 | 1145.7 |

TABLE 1i
MCP-1 in HACaT cell supernatants after 24 h in pg/l

| | | | Azelaic | Aza + |
|---|---|---|---|---|
| | Control | Control | acid | Green |
| | unirradiated | irradiated | irradiated | irradiated |
| | 24 h (n = 3) | 24 h (n = 3) | 24 h (n = 3) | 24 h (n = 3) |
| Mean | 1866.4 | 1177.8 | 683.2 | 32.9 |
| #1 | 1588.4 | 1260.3 | 619.9 | 38.7 |
| #2 | 1904.9 | 1187.6 | 520.8 | 30.9 |
| #3 | 2106.1 | 1085.5 | 908.9 | 29.0 |

TABLE 1j
MCP-1 in HACaT cell supernatants after 48 h in pg/l

| | | | Azelaic | Aza + |
|---|---|---|---|---|
| | Control | Control | acid | Green |
| | unirradiated | irradiated | irradiated | irradiated |
| | 48 h (n = 3) | 48 h (n = 3) | 48 h (n = 3) | 48 h (n = 3) |
| Mean | 3641.3 | 2466.3 | 1190.1 | 78.5 |
| #1 | 3491.9 | 2876.8 | 1174.3 | 94.1 |
| #2 | 3524.3 | 2847.0 | 1116.1 | 76.5 |
| #3 | 3907.7 | 1675.3 | 1279.9 | 65.0 |

Hitherto, there has only been limited information about the anti-inflammatory efficacy of azelaic acid on skin cells. To date, there has been no information about whether the addition of titanium dioxide-coated potassium aluminium silicate has any influence on the antiinflammatory effect of azelaic acid and inhibits or enhances this effect.

It is known that some inflammatory cytokines are activated after UVB irradiation in skin cells. This supports the clinical observations that UV radiation is a trigger for the inflammatory events in rosacea.

The induction of the inflammatory cytokines (IL-1β, IL-6, TNF-α) observed here after irradiation of keratinocytes can be reduced by addition of azelaic acid. This antiinflammatory effect was also studied by Mastrofrancesco et al., who attributed this activity inter alia to the action of azelaic acid on the intracellular receptor PPARy. The results observed in the present invention correspond essentially with the observations made by Mastrofrancesco. However, there the protein induction could be observed even 24 h after irradiation, which may possibly be due to the different nature of the keratinocytes used (HaCaTs in our work and freshly isolated neonatal foreskin keratinocytes in the case of Mastrofrancesco).

Surprisingly, it was found that there was a further marked reduction in cytokine release in the cultures treated with azelaic acid and titanium dioxide-coated potassium aluminium silicate 3%. At the time of 48 h, this was observed for all cultures in comparison with those treated only with azelaic acid. Here, IL-1β was reduced by a further 65%, IL-6 by 52%, TNF-α by 84%, VEGF by 70% and MCP-1 by 93%.

The titanium dioxide-coated potassium aluminium silicate-enhanced reduction of the expression of VEGF may be of particular significance since, in addition to its pro-inflammatory action, VEGF also contributes directly to vascular changes in rosacea (Smith J R, Lanier V B, Braziel R M, Falkenhagen K M, White C, Rosenbaum J T. Expression of vascular endothelial growth factor and its receptors in rosacea. Br J Ophthalmol. 2007; 91:226-9).

The vascular endothelial growth factor/vascular permeability factor (VEGF/VPF) is an important molecule which unfolds its activity in angiogenesis and has a mitogenic effect on endothelial cells. In addition, it also increases the permeability of the blood vessels. It stimulates in particular the endothelium of the blood vessels.

Rosacea is characterized inter alia by increased and enlarged vessels in particular in the convex portions of the face. Initially, there are in particular facial flushes and burning erythemas. The cause is assumed to be degenerative changes of collagen fibres and elastic fibres which can be triggered by repeated vasodilations. As the disorder progresses, increased vascular permeability may lead to a chronic lymphoedema of the skin (Gomaa et al.). A central pathogenetic factor which may initially be involved in the pathogenesis is VEGF or its ligands, VEGF receptors1 (Flt-1) & 2 (Flk-1).

It is known that VEGF is elevated significantly in the lesioned dermis of rosacea patients compared to skin without any lesions (Gomaa A. H. A. et al. Lymphangiogenesis and angiogenesis in non-phymatous rosacea. J Cut Pathol 2007:34:748-753). The expression of the VEGF receptors VEGF-R1 & 2 is likewise increased in the endothelium of patients suffering from rosacea (Smith J R, Lanier V B, Braziel R M, Falkenhagen K M, White C, Rosenbaum J T. Expression of vascular endothelial growth factor and its receptors in rosacea. Br J Ophthalmol. 2007; 91:226-9). The authors speculate that VEGF or its receptors may contribute to the vascular changes and thus to the pathogenesis of rosacea.

Retinoids, which include, for example, all-trans retinoic acid and isotretinoin, can reduce the VEGF production of keratinocytes in vitro (Lachgar S et al. Dermatol 1999:199 (suppl 1): 25-27). Whether this is the reason for their good efficacy in particular in cases of moderate and severe rosacea (Sobottka A, Lehmann P. Rosazea 2009: Neue Erkenntnisse zur Pathophysiologie, klinische Manifestationsformen and Therapiestrategien [Rosacea 2009: Novel insights into pathophysiology, clinical manifestations and therapeutic strategies]. Hautarzt. 2009; 60:999-1009) is as yet unclear.

Hitherto, it has not been known that azelaic acid, too, leads to a reduction of this important endothelial growth factor. Thus, this effect may also contribute to the action of azelaic acid on rosacea. In our studies, we were able to demonstrate that the VEGF production of keratinocytes is increased by the combination of azelaic acid with titanium dioxide-coated potassium aluminium silicate compared to a monotherapy. This may lead to an improved efficacy in the treatment of rosacea by combination therapy compared to monotherapy.

To summarize: it can be demonstrated that a combination of azelaic acid 20 mM and titanium dioxide-coated potassium aluminium silicate 3% has a markedly higher anti-inflammatory effect than azelaic acid 20 mM alone in UVB-irradiated skin cell cultures. It can therefore be assumed that addition of titanium dioxide-coated potassium aluminium silicate does not negatively affect the efficacy of azelaic acid in the treatment of rosacea.

On the contrary, in an unexpected manner, in addition to the positive cosmetic covering effect, the anti-inflammatory, vessel-sealing and antiproliferative action may be supported.

1. A pharmaceutical composition, comprising 1-30% azelaic acid or an azelaic acid derivative and 0.01-10% of an interference pigment.

2. A pharmaceutical composition according to claim 1, characterized in that it is a gel.

3. A pharmaceutical composition according to claim 1, characterized in that the interference pigment is a titanium dioxide-coated silicate.

4. A pharmaceutical composition according to claim 1, characterized in that the interference pigment consists of a phyllosilicate (KH₂(AlSiO₄)₃, mica) which is coated with titanium dioxide,
where phyllosilicate and titanium dioxide are present in a ratio of from 0.75:1 to 1:0.75,
where 80% of the particles have a particle size in the range of 10-60 μm and where the mean particle diameter (D₅₀) is 18-25 μm.

5. A pharmaceutical composition according to claim 1 in the form of a gel, characterized by the following composition:

| | | |
|---|---|---|
| azelaic acid | 1.00-30.00% | by weight |
| mono/diglycerides | 0.00-3.00% | by weight |
| cetostearyl alcohol | 0.00-3.00% | by weight |
| PEG-40 stearate | 0.00-5.00% | by weight |
| medium-chain triglycerides | 0.00-15.00% | by weight |
| polysorbate 80 | 0.00-3.00% | by weight |
| lecithin | 0.00-10.00% | by weight |
| mono/diglycerides/cetostearyl alcohol/ | 0.00-10.00% | by weight |
| cetyl palmitate/cocoglycerides | | |
| mono/diglycerides/ | 0.00-10.00% | by weight |
| PEG-30 glyceryl stearate | | |
| cetearyl octanoate | 0.00-3.00% | by weight |
| methylcellulose | 0.00-1.00% | by weight |
| xanthan gum | 0.00-1.00% | by weight |
| Carbopol 980 | 0.50-2.00% | by weight |
| Carbopol 971 | 0.50-2.00% | by weight |
| water | 30.00-95.00% | by weight |
| dimethyl isosorbide | 0.00-15.00% | by weight |
| propylene glycol | 5.00-15.00% | by weight |
| glycerol 85% | 0.00-15.00% | by weight |
| benzoic acid | 0.05-1.00% | by weight |
| disodium EDTA | 0.00-0.50% | by weight |
| interference pigment | 0.01-10.00% | by weight |
| Candurin ® “Green Shimmer” | | |
| sodium hydroxide | 0.00-1.00% | by weight. |

6. A pharmaceutical composition according to claim 1 in the form of a cream, characterized by the following composition:

| | | |
|---|---|---|
| azelaic acid | 1.00-30.00% | by weight |
| mono/diglycerides | 0.00-3.00% | by weight |
| cetostearyl alcohol | 0.00-3.00% | by weight |
| PEG-40 stearate | 0.00-5.00% | by weight |
| medium-chain triglycerides | 0.00-15.00% | by weight |
| polysorbate 80 | 0.00-3.00% | by weight |
| lecithin | 0.00-10.00% | by weight |
| mono/diglycerides/cetostearyl alcohol/ | 0.00-10.00% | by weight |
| cetyl palmitate/cocoglycerides | | |
| mono/diglycerides/ | 0.00-10.00% | by weight |
| PEG-30 glyceryl stearate | | |
| cetearyl octanoate | 0.00-3.00% | by weight |
| methylcellulose | 0.00-1.00% | by weight |
| xanthan gum | 0.00-1.00% | by weight |
| Carbopol 980 | 0.50-2.00% | by weight |
| Carbopol 971 | 0.50-2.00% | by weight |
| water | 30.00-95.00% | by weight |
| dimethyl isosorbide | 0.00-15.00% | by weight |
| propylene glycol | 5.00-15.00% | by weight |
| glycerol 85% | 0.00-15.00% | by weight |
| benzoic acid | 0.05-1.00% | by weight |
| disodium EDTA | 0.00-0.50% | by weight |
| interference pigment | 0.01-10.00% | by weight |
| Candurin ® “Green Shimmer” | | |
| sodium hydroxide | 0.00-1.00% | by weight |

7. A pharmaceutical composition according to claim 1 in the form of a lotion, characterized by the following composition:

| | | |
|---|---|---|
| azelaic acid | 1.00-30.00% | by weight |
| mono/diglycerides | −0.00-3.00% | by weight |
| cetostearyl alcohol | 0.00-3.00% | by weight |
| PEG-40 stearate | 0.00-5.00% | by weight |
| medium-chain triglycerides | 0.00-15.00% | by weight |
| polysorbate 80 | 0.00-3.00% | by weight |
| lecithin | 0.00-10.00% | by weight |
| mono/diglycerides/cetostearyl alcohol/ | 0.00-10.00% | by weight |
| cetyl palmitate/cocoglycerides | | |
| mono/diglycerides/ | 0.00-10.00% | by weight |
| PEG-30 glyceryl stearate | | |
| cetearyl octanoate | 0.00-3.00% | by weight |
| methylcellulose | 0.00-1.00% | by weight |
| xanthan gum | 0.00-1.00% | by weight |
| Carbopol 980 | 0.50-2.00% | by weight |
| Carbopol 971 | 0.50-2.00% | by weight |
| water | 30.00-95.00% | by weight |
| dimethyl isosorbide | 0.00-15.00% | by weight |
| propylene glycol | 5.00-15.00% | by weight |
| glycerol 85% | 0.00-15.00% | by weight |
| benzoic acid | 0.05-1.00% | by weight |
| disodium EDTA | 0.00-0.50% | by weight |
| interference pigment | 0.01-10.00% | by weight |
| Candurin ® “Green Shimmer” | | |
| (mica + TiO₂) | | |
| sodium hydroxide | 0.00-1.00% | by weight. |

8. A pharmaceutical composition according to claim 1 in the form of a foam, characterized by the following composition:

| | | |
|---|---|---|
| azelaic acid | 1.00-30.00% | by weight |
| mono/diglycerides | 0.00-3.00% | by weight |
| cetostearyl alcohol | 0.00-3.00% | by weight |
| PEG-40 stearate | 0.00-5.00% | by weight |
| medium-chain triglycerides | 0.00-15.00% | by weight |
| polysorbate 80 | 0.00-3.00% | by weight |
| lecithin | 0.00-10.00% | by weight |
| mono/diglycerides/cetostearyl alcohol/ | 0.00-10.00% | by weight |
| cetyl palmitate/cocoglycerides | | |
| mono/diglycerides/ | 0.00-10.00% | by weight |
| PEG-30 glyceryl stearate | | |
| cetearyl octanoate | 0.00-3.00% | by weight |
| methylcellulose | 0.00-1.00% | by weight |
| xanthan gum | 0.00-1.00% | by weight |
| Carbopol 980 | 0.50-2.00% | by weight |
| Carbopol 971 | 0.50-2.00% | by weight |
| water | 30.00-95.00% | by weight |
| dimethyl isosorbide | 0.00-15.00% | by weight |
| propylene glycol | 5.00-15.00% | by weight |
| glycerol 85% | 0.00-15.00% | by weight |
| benzoic acid | 0.05-1.00% | by weight |
| disodium EDTA | 0.00-0.50% | by weight |
| interference pigment | 0.01-10.00% | by weight |
| Candurin ® “Green Shimmer” | | |
| sodium hydroxide | 0.00-1.00% | by weight |

9-11. (canceled)

12. A medicament for the therapy of rosacea and acne, comprising a composition according to claim 1.

13. A process for preparing a composition according to claim 1, comprising the steps of:
a) mixing of the hydrophilic components,
b) heating to 60°-80° C.,
c) dissolving of preservatives and complex formers in the warm mixture,
d) addition and dispersion of the pigment or the pigments by intensive homogenization,
e1) addition and dispersion of the gel former or the gel formers by another homogenization,
e2) heating of the oil phase to 60°-80° C., in parallel to step e1
f) addition of the oil phase from e2 to the prepared dispersion from e1 at 60-80° C., and another homogenization of the overall formulation,
g) cooling to 20°-35° C.,
h) addition of azelaic acid to the dispersion (cooled to 20°-35° C.), with avoidance of heating and/or sheering,
i) pH-stabilization with formation of the gel by addition of sodium hydroxide solution to a pH of 4.6-5.0.
